(19) United States
(12) Plant Patent Application Publication
(10) Pub. No.: US 2003/0208819 P1
(43) Pub. Date: Nov. 6, 2003

(54) CHRYSANTHEMUM PLANT NAMED 'GEDI TWO RAN'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/137,803

(22) Filed: May 1, 2002

(51) Int. Cl. A01H 5/00

(52) U.S. Cl. PLT/293

(57) ABSTRACT

A new and distinct Chrysanthemum plant cultivar is disclosed, characterized by a decorative-type inflorescence, consistent flowering response to short days, blooming consistently after 50 days of short day length, free branching habit, red ray florets, a very uniform round growth habit, and a large quantity of blooms per flowering branch.

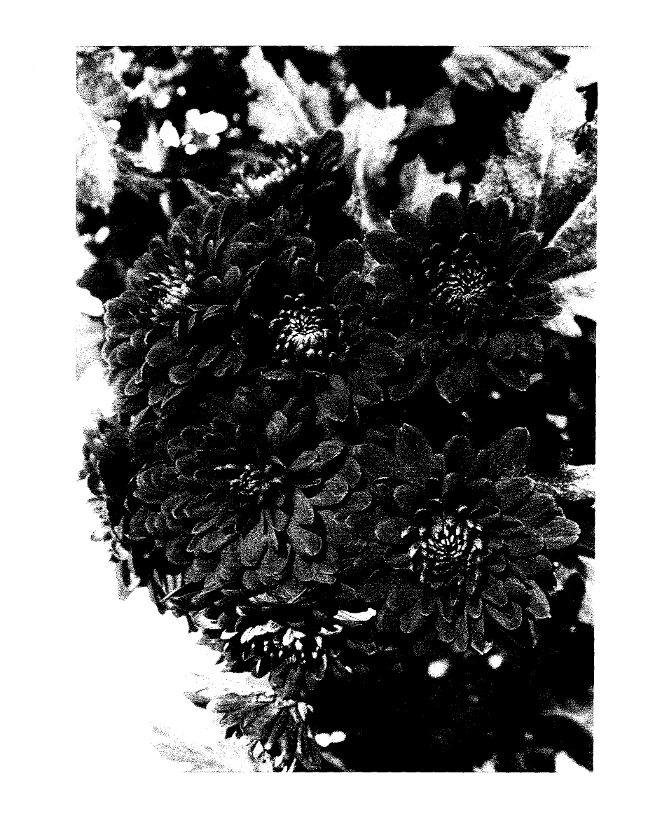

### LATIN NAME OF THE GENUS AND SPECIES

The present invention relates to a new and distinct cultivar of Chrysanthemum plant, botanically known as Dendranthema morifolium.

### VARIETY DENOMINATION

The new and distinct Chrysanthemum plant is hereinafter referred to by the cultivar name ‘GEDI TWO RAN.’

### BACKGROUND OF THE INVENTION

The new cultivar is a product of hybridization of a female parent variety, ‘GEDI TWO CAM’ (United States plant patent pending) and a male parent variety ‘Medonia Red’ (undistributed in the United States). The new cultivar was discovered and selected by the inventor in October 1998.

Asexual reproduction of the new cultivar by apical tip cutting and meristem tissue culture was performed in Oxnard, Calif. and has shown that the unique features of this cultivar are stable and reproduced true to type on successive generations.

### SUMMARY OF THE INVENTION

The cultivar ‘GEDI TWO RAN’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI TWO RAN.’ These characteristics in combination distinguish ‘GEDI TWO RAN’ as a new and distinct Chrysanthemum cultivar:

1. Decorative-type inflorescence,

2. Consistent flowering response to short days, blooming consistently after 50 days of short day length,

3. Free branching habit,

4. Red ray florets,

5. Very uniform round growth habit, and

6. Large quantity of blooms per flowering branch.

Plants of the new cultivar are similar to plants of the female parent variety, ‘GEDI TWO CAM’ in most horticultural characteristics, however plants of the new cultivar have more ray florets per inflorescence and inflorescences with a smaller diameter than the female parent variety. Plants of the new cultivar also bloom naturally two weeks earlier than plants of the female parent variety.

Plants of the new cultivar are similar to plants of the male parent variety, ‘Medonia Red,’ however the new cultivar has ray florets which are a lighter red color than plants of the male parent variety. Plants of the new cultivar also bloom naturally three weeks earlier than plants of the male parent variety, and plants of the new cultivar have fewer inflorescences per flowering branch than plants of the ‘Medonia Red’ variety.

In comparison to the commercially available variety ‘Jenny Wren’ (U.S. Plant Pat. No. 10,213), ‘GEDI TWO RAN’ has ray florets of a different color, fewer ray florets per inflorescence, leaves that are more than twice as large, and more blooms per flowering branch.

### BRIEF DESCRIPTION OF THE PHOTOGRAPH

The photograph shows a plant of ‘GEDI TWO RAN’ grown in a six-inch container. One cutting was used in the pot.

### DETAILED BOTANICAL DESCRIPTION

In the following description, color references are made to The Royal Horticultural Society Colour Chart except where general terms of ordinary dictionary significance are used. The following observations and measurements describe plants grown in Oxnard, Calif. during the month of December and January. The growing temperature ranged from 55° to 68° F. at night to 60° to 75° F. during the day. Measurements and numerical values represent averages of typical flowering types.

Botanical classification: Dendranthema morifolium cultivar ‘GEDI TWO RAN’.

Commercial classification: Garden-type Chrysanthemum.

Parentage: Hybridization of a female parent variety, ‘GEDI TWO CAM’ (United States plant patent pending), and a male parent variety ‘Medonia Red’ (undistributed in the United States).

### PROPAGATION

Time to rooting: 7 to 14 days at approximately 21° C.

Rooting habit: Fine, fibrous.

### PLANT

Growth habit: Mounding herbaceous perennial.

Height: Approximately 20 cm.

Spread: Approximately 22 cm.

Growth rate: Moderate.

Branching characteristics: Free Branching.

Length of lateral branches: Approximately 20.5 cm.

Number of leaves per lateral branch: Approximately 53.

### FOLIAGE

Leaf:

Arrangement.—Alternate.

Average length.—Approximately 6.5 cm.

Average width.—Approximately 5.1 cm.

Shape of blade.—Ovate.

Apex.—Cuspidate.

Base.—Attenuate.

Attachment.—Stalked.

Margin.—Palmately lobed.

Texture of top surface.—Lightly pubescent.

Texture of bottom surface.—Lightly pubescent.

Color.—Young foliage upper side: Near 137B. Young foliage under side: Near 138B. Mature foliage upper side: Near 137B. Mature foliage under side: Near 138B. Venation upper side: Near 145A. Venation underside: Near 138C.

Type.—Net.

Petiole:

Average length.—Approximately 2.8 cm.

Color.—Near 137B.

Diameter.—Approximately 0.3 cm.

### BLOOM

Inflorescence:

Flowering habit.—Induced by darkness period greater than 13.5 hours, approximately 50 days of appropriate day length required to induce and develop blooms.

Inflorescence form.—Decorative.

Natural flowering season.—Approximately the first two weeks of September.

Number of inflorescences per lateral branch.—Approximately 14.

Inflorescence diameter.—Approximately 4 cm.

Inflorescence depth.—Approximately 1.5 cm.

Inflorescence longevity on plant.—Approximately 15 days.

Persistence.—Persistent.

Ray florets:

Appearance.—Matte.

Texture.—Smooth.

Average number per flower.—153.

Shape.—Oblong.

Aspect.—Flat.

Margin.—Entire.

Apex.—Retuse.

Length.—Approximately 2.2 cm.

Width.—Approximately 0.8 cm.

Color.—Upper surface at first opening: Near 185A. Upper surface at maturity: Near 46A. Upper surface at fading: Near 179A. Under surface at first opening: Near 182B. Under surface at maturity: Near 181D. Under surface at fading: Near 179C.

Disc florets:

Appearance.—Shiny.

Texture.—Smooth.

Average number per flower.—Approximately 3.

Shape.—Cylindric.

Apex.—Obtuse.

Average length.—Approximately 0.3 cm.

Average width.—Approximately 0.1 cm.

Color.—At first opening: Near 154B. At maturity: Near 2B. At fading: Near 6A.

Peduncle:

Length.—At terminal end (shortest): Approximately 2.6 cm. At lateral end (longest): Approximately 9 cm.

Angle to stem.—Acute.

Strength.—Moderate.

Color.—Near 138B.

Habit.—Upright.

Diameter.—Approximately 0.2 cm.

Inflorescence bud:

Length.—Approximately 0.7 cm.

Diameter.—Approximately 0.7 cm.

Form.—Globular.

Color.—Near 187C.

Involucral bracts (Phyllaries):

Appearance.—Matte.

Texture.—Lightly pubescent.

Number.—Approximately 25.

Shape.—Oblanceolate.

Margin.—Entire.

Apex.—Acute.

Length.—Approximately 0.7 cm.

Width.—Approximately 0.4 cm.

Color.—Upper side: Near 143A. Under side: Near 138A.

### REPRODUCTIVE ORGANS

Ray florets:

Pistillate.—Number of pistils per flower: 1. Stigma shape: 2 branched. Stigma color: Near 1A. Style color: Near 1C. Style length: Approximately 0.4 cm.

Disc florets (hermaphroditic):

Pistillate.—Number of pistils per flower: 1. Stigma shape: Two-branched. Stigma color: Near 2A. Style length: Approximately 0.4 cm. Style color: Near 154C.

Staminate.—Number of stamens per flower: 5. Anther shape: Tubular. Anther color: Near 15A.

Pollen.—No pollen detected.

What is claimed is:

1. A new and distinct cultivar of Chrysanthemum plant named ‘GEDI TWO RAN’ as herein illustrated and described.
